(19) United States
(12) Patent Application Publication
(10) Pub. No.: US 2014/0081221 A1
(43) Pub. Date: Mar. 20, 2014

(54) ANTISEPTIC APPICATOR HAVING HYDROPHOBIC FOAM FOR USE WITH ANTISEPTIC SOLUTIONS

(71) Applicants: James Patrick MCDONALD, El Paso, TX (US); Hugo Chavez, El Paso, TX (US); Kenneth M. Dokken, El Paso, TX (US); James Bardwell, El Paso, TX (US)

(72) Inventors: James Patrick MCDONALD, El Paso, TX (US); Hugo Chavez, El Paso, TX (US); Kenneth M. Dokken, El Paso, TX (US); James Bardwell, El Paso, TX (US)

(73) Assignee: CAREFUSION 2200, INC., San Diego, CA (US)

(21) Appl. No.: 13/619,838

(22) Filed: Sep. 14, 2012

(51) Int. Cl. A61M 35/00

(52) U.S. Cl. CPC A61M 35/003
USPC 604/290; 401/196

(57) ABSTRACT

An applicator having a hydrophobic foam that is suitable for use with antiseptic solutions may be a hydrophobic polyurethane foam that does not reduce the amount of active ingredient in the solution as it passes through the foam so that it can be applied to the skin. The foam may be used to deliver antiseptics solutions such as octenidine salts, chlorhexidine salts, or any cationic molecule to the skin. The solution may include alcohol solution. The foam may be attached to an applicator containing an antiseptic solution, or it may be used in conjunction with an unattached antiseptic solution reservoir. The foam also allows the solution to wick into the foam and fully saturate the foam without dripping or channeling.

### FIELD OF THE INVENTION

The invention relates to an antiseptic applicator having a foam suitable for use with antiseptic solutions and methods for using the antiseptic applicator. The foam may be made of a hydrophobic material that does not reduce the amount of active ingredient in an antiseptic solution as it passes through the foam.

### BACKGROUND OF THE INVENTION

Solutions containing antiseptics, such as octenidine and chlorhexidine, may be applied to the skin to kill bacteria on the skin before performing a medical procedure. Typically, an antiseptic solution is applied to the skin using an antiseptic applicator having a foam pad, which may be attached to the applicator that contains the antiseptic solution. Generally, when an antiseptic solution comes into contact with medical-grade foams, the active component of the solution, such as octenidine and chlorhexidine, often interacts with the foam and may bond to the foam. This interaction reduces the amount of the active component that is available to kill bacteria on the skin.

Thus, there is a need for an antiseptic applicator having a foam that does not reduce the amount of the available active antiseptic component in the solution.

### SUMMARY OF THE INVENTION

The invention relates to an antiseptic applicator having a hydrophobic foam suitable for use with antiseptic solutions. The foam comprises a hydrophobic polyurethane foam that does not substantially reduce the amount of active ingredient in the solution as it passes through the foam so that it can be applied to the skin. The foam may be used to deliver antiseptic solutions including, but not limited to octenidine salts, chlorhexidine salts, aldehyde, anilide, diamidine, halogen-releasing agent, silver compound, peroxygen, and or phenols and/or biguanides such as alexidine, phenoxyethanol, bis-phenols, quaternary ammonium compounds, PVP-iodine, or any cationic molecule to the skin. The foam may also be used in conjunction with a separate container comprising antiseptic solution instead of being attached to an applicator. The foam also allows the solution to wick into the foam and fully saturate the foam without dripping or channeling.

### DETAILED DESCRIPTION OF THE INVENTION

The invention relates to an antiseptic applicator having a hydrophobic foam suitable for use with antiseptic solutions. The foam comprises a hydrophobic polyurethane foam that does not substantially reduce the amount of active ingredient in the solution as it passes through the foam so that it can be applied to the skin. The foam may be used to deliver antiseptics solutions such as octenidine salts, chlorhexidine salts, alcohol, aldehyde, anilide, diamidine, halogen-releasing agent, silver compound, peroxygen, and or phenols or biguanides such as alexidine, phenoxyethanol, bis-phenols, quaternary ammonium compounds, PVP-iodine any cationic molecule to the skin.

Preferably, the solution is an alcohol solution, for example containing 0.001%-10% of antiseptic. When the antiseptic solution comes in contact with the foam, the properties of the foam allow the solution to quickly wick into the foam and fully saturate the foam to create a reservoir of solution.

According to an embodiment of the invention, the foam may comprise a hydrophobic, non-swelling, biocompatible foam, preferably comprising polyester urethane. A preferred hydrophobic foam that may be used in this invention is Foamex Clear #3-1000Z felt, (Natural Color Non-Pigmented) Reticulated Polyester Urethane obtained from FXI Inc. The hydrophobic foam may have one or more of the following qualities: a density of 1.7-2.2 lbs/ft³, a tensile strength of 15 psi-45 psi, an elongation of 200-300%, a compression load deflection of 0.02-0.04 psi at 25% deflection, a percent compression set of 15-25%, and a wicking rate of 0.5-6.5 sec. According to the invention, this foam may be used with an alcohol antiseptic solution preferably containing an active ingredient such as octenidine dihydrochloride or chlorhexidine gluconate.

The foam is configured to allow 90% or more of the antiseptic to remain in the solution after the solution passes through the hydrophobic foam member, preferably 95% or more, and most preferably 98% or more.

A foam according to the invention may be cut into various shapes and adapted to fit a delivery device. For example, a foam may be used as an applicator for a surgical preparation applicator used to prepare a surgical site or placed over a wound to promote healing. The antiseptic solution may be contained in a reservoir, such as a frangible ampoule, inside the applicator.

An applicator according to the invention may comprise a head portion and a container portion. The head portion includes a proximal end, a distal end, and an interior portion defining a fluid chamber. The container portion may include a reservoir that contains the antiseptic solution. The foam member may be attached to the distal end. The foam and antiseptic solution of the invention may be implemented in any suitable applicator apparatus. Some examples of suitable applicators include: U.S. Pat. Nos. 3,757,782; 4,415,288; 4,498,796; 5,288,159; 5,308,180; 5,435,660; 5,445,462; 5,658,084; 5,772,346; 5,791,801; 5,927,884; 6,371,675; 5,769,552; 6,488,665; 6,916,133; 7,201,525; and 7,866,907, each hereby incorporated by reference herein.

Alternatively, the foam of the invention may be used in conjunction with unattached separate container comprising antiseptic solution.

When applying an antiseptic solution to the skin, it may be desirable to include a dye with the antiseptic solution so that the areas of the skin contacted with the antiseptic solution may be visible to a user. This dye may be incorporated into the antiseptic solution, or it may be incorporated into a pledget, which is a piece of material typically made of a foam or a laminate. The pledget may be made from 100% non-woven needle-punched polyester pledgets having a fiber weight per unit length of 6 denier. In an embodiment of the invention, the foam may be used with an applicator that includes a pledget containing a dye. As the antiseptic solution flows out of its reservoir in the applicator, the antiseptic solution passes through the pledget, and the antiseptic solution and dye pass on to the foam. Examples of such an arrangement can be found in U.S. Pat. No. 7,866,907 and U.S. application Ser. No. 12/961,255, filed on Dec. 6, 2010, each hereby incorporated by reference.

In accordance with the present specification, the term “substantially” means preferably about 10% or less, more preferably about 5% or less.

Experiments were performed to compare the performances of various types of foam when contacted with an alcohol solution containing about 0.2% w/v octenidine hydrochloride. The experiments measured physical flow characteristics and the percent loss of octenidine after the antiseptic solution passed through the foam.

The foams tested were Foamex (FXI) clear 3-1000Z, Crest clear, Woodbridge Foam Partner (WFP) clear, INOAC clear, and Crest orange. FXI clear foam (specifically part # FLM141) comprises a natural color non-pigmented polyester urethane. The FXI clear foam has a pore size of 85 ppi, a density of 1.93 lb/ft³, a tensile strength of 40 psi minimum, an elongation 200% minimum, and a laminated bond strength of 16 oz minimum. INOAC clear comprises a plastic made of hydroxyl compounds (polyol) and isocyanate. INOAC clear has a density 32.7 kg/m³, a hardness 0.65 psi, a tensile strength of 15 lb/in, a pore count of 62-66 ppi, an elongation of 250%, a wicking rate of 2-3 seconds, an absorption of 18 times, and an expansion 3%. Crest foam has the same specifications as INOAC foam, but is manufactured in a different manufacturing facility. Crest orange have the same specifications as Crest clear, but orange dye is added to the pledget. WFP foam comprises thermally reticulated polyester. WFP has a density of 1.7-2.1 pounds per cubic foot, a compression load deflection of 0.4-0.6 lbf @ 25%, a tensile strength of 30 psi minimum, an elongation of 240% minimum, and a pore count of 75-90 ppi. Three to five samples of each type of foam were tested.

Physical parameters including wet time, drip time, and drain time after activation, or when the antiseptic solution was released from its reservoir, were measured for each of the foams tested. The wet time is the time for the fluid to reach the foam and passes through the foam and is visible on the bottom or other side from the ampoule side, starting from the time of activation. A foam that achieves a wet time of 10 seconds or less is preferable, and a wet time of 5 seconds or less is more preferable. The drip time is the amount of time after activation at which the foam tip begins to drip. A foam that exhibits no dripping within 15 seconds of activation is preferable. In the experiments, if a foam did not exhibit any dripping within 15 seconds of activation, the drip time was recorded as “no drip” (ND). This time is also referred herein as the dripping termination time. The drain time is the time that takes the applicator to discharge the entire solution into the foam. A foam that exhibits drain time of less than 30 seconds is preferable.

Another test called “extraction” or “milking” was performed by forcing the antiseptic solution through the foam to determine how much of the solution may be extracted from the foam. This test was performed by weighing the applicator containing the antiseptic solution and foam, recording the initial weight of the applicator, placing the applicator inside a glass beaker, activating the applicator by opening the reservoir, waiting 30 seconds for the antiseptic solution to drain into the foam tip, extracting the antiseptic solution by pressing on the foam tip against inner wall of the glass beaker for 30 seconds to force solution out of foam tip, removing the applicator from the glass beaker, weighing the applicator, and recording the final weight of the applicator.

The extraction amount was calculated by subtracting the final weight of the applicator from the initial weight of the applicator. Preferably, the foam achieves an extraction amount evidencing that about 90% or more of the antiseptic solution, more preferably about 95% or more, most preferably about 98% or more, passes through the foam.

The concentration of antiseptic in the solutions before and after passing through the foams was measured by high pressure liquid chromatography (HLPC) performed on an Agilent 1200 HPLC-6 instrument with a Zorbax Eclipse XDB-C18 3.0×150 mm, 5 micron column.

The percent loss of octenidine for each foam, which is based on the ratio of concentration of the octenidine in the solution extracted from the foam and the concentration of the octenidine in the antiseptic solution before passing through the foam, was calculated according to the Formula 1:

$$Loss of Octenidine ( \% ) = 1 - \frac{\begin{matrix}octenidine concentration in \\ solution extracted from foam\end{matrix}}{\begin{matrix}octenidine concentration in \\ solution before passing through foam\end{matrix}} \times 100 \qquad Formula 1$$

As each foam sample was tested three to five times, an average percent loss of octenidine for each type of foam was calculated.

Applicators having reservoirs of 3 mL, 10.5 mL and 26 mL sizes were tested. The 3 mL applicators had circular shape foams having a diameter of 1.5 inches and a thickness of 0.330 inches. The 10.5 mL applicators had circular-shaped foams having a diameter of 1.5 inches and a thickness of 0.360 inches. The 26 mL applicators had square-shaped foams having a side length of 2.125 inches and a thickness of 0.360 inches.

In the experiments, the applicators included different types of pledgets that were embedded with a dye. In some experiments, 100% non-woven needle-punched polyester pledgets having a fiber weight per unit length of 6 denier were used. Applicators having these pledgets are denoted as “PLP” in the table below.

In other experiments, the pledgets were formed of a laminated material formed of Novonette SP-149305, a nonwoven polyester, and SIF #3-1000Z felt, a non-pigmented reticulated polyester urethane. Applicators having these pledgets are denoted as “FOP” in the table below. The FOP has a thickness of approximately 0.593 inches.

In other experiments, the pledgets were a thin foam pledget (TFP), which were formed from a laminated material formed of Novonette SP-149305, a nonwoven polyester, and SIF #3-1000Z felt, a non-pigmented reticulated polyester urethane, and have approximately half the thickness of the “FOP” pledget. The TFP has a thickness of approximately 0.360 in. Applicators having these pledgets are denoted as “TFP” in the table below.

The experimental results are summarized in Table 1 below.

TABLE 1

| | | | | | | | | Loss |
|---|---|---|---|---|---|---|---|---|
| | | Wet | Drip | Drain | | | Average | of |
| | Pledget | Time | Time* | Time | Extraction | OCT % | OCT % | OCT % |
| Foam | Type | (sec) | (sec) | (sec) | (grams) | (w/v) | (w/v) | (w/v) |
| Baseline IPA/OCT | PD1023 | N/A | N/A | N/A | 24.0 mL | 0.19984 | 0.19974 | 0% |
| (No foam) | | | | | | | | |
| Baseline IPA/OCT | PD1023 | N/A | N/A | N/A | 23.5 mL | 0.19944 | | |
| (No foam) | | | | | | | | |
| Baseline IPA/OCT | PD1023 | N/A | N/A | N/A | 22.5 mL | 0.19994 | | |
| (No foam) | | | | | | | | |
| Foamex 26 mL | FOP | 2.67 | ND | 37.92 | 14.80 | 0.20535 | 0.20403 | 0% |
| clear | | | | | | | | |
| Foamex 26 mL | FOP | 2.70 | ND | 38.03 | 14.78 | 0.20314 | | |
| clear | | | | | | | | |
| Foamex 26 mL | FOP | 2.85 | ND | 42.27 | 14.44 | 0.20361 | | |
| clear | | | | | | | | |
| Foamex 26 mL | PLP | 1.63 | ND | 11.16 | 13.13 | 0.20368 | 0.20312 | 0% |
| clear | | | | | | | | |
| Foamex 26 mL | PLP | 2.68 | ND | 12.48 | 13.71 | 0.20276 | | |
| clear | | | | | | | | |
| Foamex 26 mL | PLP | 2.87 | ND | 13.33 | 13.11 | 0.20291 | | |
| clear | | | | | | | | |
| Crest 26 mL clear | FOP | 4.22 | ND | 28.78 | 13.25 | 0.20052 | 0.20149 | 0% |
| Crest 26 mL clear | FOP | 2.93 | ND | 28.24 | 14.34 | 0.20234 | | |
| Crest 26 mL clear | FOP | 3.43 | ND | 32.09 | 14.44 | 0.20161 | | |
| WFP 26 mL clear | FOP | 2.35 | ND | 23.21 | 14.30 | 0.19969 | 0.19814 | 1% |
| WFP 26 mL clear | FOP | 2.62 | ND | 7.22+ | 13.90 | 0.19549 | | |
| WFP 26 mL clear | FOP | 2.68 | ND | 15.46+ | 14.82 | 0.19923 | | |
| INOAC 26 mL clear | FOP | 3.06 | ND | 32.74 | 12.20 | 0.18668 | 0.18750 | 6% |
| INOAC 26 mL clear | FOP | 2.02 | ND | 4.20+ | 13.30 | 0.18580 | | |
| INOAC 26 mL clear | FOP | 3.52 | ND | 38.44 | 13.27 | 0.19003 | | |
| Crest 10.5 mL | PLP | 2.39 | ND | 9.89 | 4.48 | 0.11546 | 0.09673 | 52% |
| orange | | | | | | | | |
| Crest 10.5 mL | PLP | 4.78 | ND | 11.81 | 4.80 | 0.08507 | | |
| orange | | | | | | | | |
| Crest 10.5 mL | PLP | 4.98 | ND | 15.79 | 5.49 | 0.08966 | | |
| orange | | | | | | | | |
| Foamex 26 mL | No P | 1.10 | ND | 3.39 | 13.46 | 0.20210 | 0.20220 | 0% |
| clear | | | | | | | | |
| Foamex 26 mL | No P | 0.82 | ND | 2.78 | 16.07 | 0.203750 | | |
| clear | | | | | | | | |
| Foamex 26 mL | No P | 0.68 | ND | 1.85 | 15.31 | 0.20076 | | |
| clear | | | | | | | | |
| Crest 10.5 mL clear | TFP | 1.57 | 2 | 8.70 | 5.27 | 0.20376 | 0.20095 | 0% |
| | | | drops | | | | | |
| Crest 10.5 mL clear | TFP | 2.39 | Drip | 8.59 | 5.52 | 0.19958 | | |
| Crest 10.5 mL clear | TFP | 2.69 | ND | 6.74 | 5.12 | 0.19951 | | |
| WFP 3 mL clear | N/A | 5.64 | ND | 12.10 | 0.98 | 0.21544 | 0.21544 | 0% |
| WFP 3 mL clear | N/A | 5.58 | ND | 9.79 | 1.86 | N/A | | |
| WFP 3 mL clear | N/A | 2.38 | ND | 5.23 | 3.20 | N/A | | |
| WFP 3 mL clear | N/A | 2.03 | ND | 4.17 | 4.60 | N/A | | |
| Crest 3 mL clear | N/A | 2.25 | ND | 4.82 | 1.28 | 0.21033 | 0.21033 | 0% |
| Crest 3 mL clear | N/A | 1.37 | ND | 3.34 | 2.41 | N/A | | |
| Crest 3 mL clear | N/A | 1.93 | ND | 4.91 | 3.69 | N/A | | |
| Crest 3 mL clear | N/A | 1.52 | ND | 4.92 | 4.74 | N/A | | |
| INOAC 3 mL clear | N/A | 1.44 | ND | 3.24 | 1.55 | 0.21214 | 0.21214 | 0% |
| INOAC 3 mL clear | N/A | 0.91 | ND | 2.53 | 2.98 | N/A | | |
| INOAC 3 mL clear | N/A | 1.40 | ND | 2.87 | 4.48 | N/A | | |
| INOAC 3 mL clear | N/A | 2.00 | ND | 4.20 | 5.89 | N/A | | |
| Foamex 3 mL clear | N/A | 1.58 | ND | 5.02 | 1.36 | 0.21308 | 0.21308 | 0% |
| Foamex 3 mL clear | N/A | 1.03 | ND | 3.23 | 2.64 | N/A | | |
| Foamex 3 mL clear | N/A | 1.58 | ND | 3.40 | 4.19 | N/A | | |
| Foamex 3 mL clear | N/A | 2.36 | ND | 4.51 | 5.53 | N/A | | |

IPA: Isopropyl Alcohol
ND: No drip
PLP: Polyester Pledget
FOP: Foam Pledget
TFP: Thin Foam Pledget
No P: No Pledget
+Foam Pledget not seated properly - the relatively low time to drain signals that the solution did not go through the pledget.

Table 1 shows that the only foam that had 0% loss of OCT on all the configurations and sizes is Foamex.

What is claimed is:

1. An applicator assembly comprising:
a head portion having a proximal end, a distal end, and an interior portion defining a fluid chamber;
a container coupled to the head portion and comprising a solution containing an antiseptic; and
a hydrophobic foam member attached to the distal end of the head portion,
wherein the hydrophobic foam member is configured to allow 90% or more of the antiseptic to remain in the solution after the solution passes through the hydrophobic foam member.

2. The applicator of claim 1, wherein the hydrophobic foam member is configured to allow 95% or more of the antiseptic to remain in the solution after the solution passes through the hydrophobic foam member.

3. The applicator of claim 1, wherein the hydrophobic foam member is configured to allow 98% or more of the antiseptic to remain in the solution after the solution passes through the hydrophobic foam member.

4. The applicator of claim 1, wherein the antiseptic is selected from the group consisting of: octenidine salts, chlorhexidine salts, alcohol, aldehyde, anilide, diamidine, halogen-releasing agent, silver compound, peroxygen, phenols, and biguanides.

5. The applicator of claim 4, wherein the solution comprises from 0.001%-10% by volume antiseptic.

6. The applicator of claim 4, wherein the antiseptic comprises octenidine hydrochloride or chlorhexidine gluconate.

7. The applicator of claim 1, wherein the solution comprises an alcohol solution.

8. The applicator of claim 1, wherein the hydrophobic foam member comprises at least one of: a density of 1.7-2.2 lbs/ft³, a tensile strength of 15 psi-45 psi, an elongation of 200-300%, a compression load deflection of 0.02-0.04 psi at 25% deflection, a percent compression set of 15-25%, and a wicking rate of 0.5-6.5 seconds.

9. The applicator of claim 1, wherein the hydrophobic foam member comprises a wet time of 10 seconds or less, a dripping termination time of 15 seconds or less, and a drain time of less than 30 seconds.

10. A solution containing system comprising:
a container comprising a solution containing an antiseptic; and
a hydrophobic foam member,
wherein the hydrophobic foam member is configured to allow 90% or more of the antiseptic to remain in the solution after the solution passes through the hydrophobic foam member.

11. The system of claim 10, wherein the hydrophobic foam member is configured to allow 95% or more of the antiseptic to remain in the solution after the solution passes through the hydrophobic foam member.

12. The system of claim 10, wherein the hydrophobic foam member is configured to allow 98% or more of the antiseptic to remain in the solution after the solution passes through the hydrophobic foam member.

13. The system of claim 10, wherein the antiseptic is selected from the group consisting of: octenidine salts, chlorhexidine salts, alcohol, aldehyde, anilide, diamidine, halogen-releasing agent, silver compound, peroxygen, phenols, and biguanides.

14. The system of claim 13, wherein the solution comprises from 0.001%-10% by volume antiseptic.

15. The system of claim 13, wherein the antiseptic comprises octenidine hydrochloride or chlorhexidine gluconate.

16. The system of claim 10, wherein the solution comprises an alcohol solution.

17. The system of claim 10, wherein the hydrophobic foam member comprises at least one of: a density of 1.75-2.10 lbs/ft³, a tensile strength of 20 psi-40 psi, an elongation of 200-300%, a compression load deflection of 0.030 psi at 25% deflection, a percent compression set of 20%, and a wicking rate of 1.0-5.0 seconds.

18. The system of claim 10, wherein the hydrophobic foam member comprises a wet time of 10 seconds or less, a dripping termination time of 15 seconds or less, and a drain time of less than 30 seconds.

19. A method of treating a surface comprising:
passing a solution containing an antiseptic through a hydrophobic foam member, wherein after passing the solution through the hydrophobic foam member, 90% or more of the antiseptic remains in the solution; and
contacting the solution with a surface.

20. The method of claim 19, wherein after passing the solution through the hydrophobic foam member, 95% or more of the antiseptic remains in the solution.

21. The method of claim 19, wherein after passing the solution through the hydrophobic foam member, 98% or more of the antiseptic remains in the solution.

22. The method of claim 19, wherein the antiseptic is selected from the group consisting of: octenidine salts, chlorhexidine salts, alcohol, aldehyde, anilide, diamidine, halogen-releasing agent, silver compound, peroxygen, phenols, and biguanides.

23. The method of claim 22, wherein the solution comprises from 0.001%-10% by volume antiseptic.

24. The method of claim 22, wherein the antiseptic comprises octenidine hydrochloride or chlorhexidine gluconate.

25. The method of claim 19, wherein the solution comprises an alcohol solution.

26. The method of claim 19, wherein the hydrophobic foam member comprises at least one of: a density of 1.75-2.10 lbs/ft³, a tensile strength of 20 psi-40 psi, an elongation of 200-300%, a compression load deflection of 0.030 psi at 25% deflection, a percent compression set of 20%, and a wicking rate of 1.0-5.0 seconds.

27. The method of claim 19, wherein the hydrophobic foam member comprises a wet time of 10 seconds or less, a dripping termination time of 15 seconds or less, and a drain time of less than 30 seconds.

28. The method of claim 19, wherein the surface comprises skin.
